(19) United States
(12) Patent Application Publication
(10) Pub. No.: US 2023/0390214 A1
(43) Pub. Date: Dec. 7, 2023

(54) ADHESIVE PATCH

(71) Applicants: TOYO INK SC HOLDINGS CO., LTD., Tokyo (JP); TOYOCHEM CO., LTD., Tokyo (JP)

(72) Inventors: Hiroto TERASHIMA, Tokyo (JP); Yutaka KATO, Tokyo (JP); Tomomi HOSHINO, Tokyo (JP); Toshikazu KOMODA, Tokyo (JP)

(73) Assignees: TOYO INK SC HOLDINGS CO., LTD., Tokyo (JP); TOYOCHEM CO., LTD., Tokyo (JP)

(21) Appl. No.: 18/021,933

(22) PCT Filed: Aug. 18, 2021
(86) PCT No.: PCT/JP2021/030188

§ 371 Date: Feb. 17, 2023

(30) Foreign Application Priority Data
Aug. 19, 2020 (JP) 2020-138958

(51) Int. Cl. A61K 9/70; A61K 31/496; A61K 31/485; A61K 31/381; A61K 31/165; A61K 47/12

(52) U.S. Cl. CPC A61K 9/7061; A61K 31/496; A61K 31/485; A61K 31/381; A61K 31/165; A61K 47/12

(57) ABSTRACT

An object is to provide an adhesive patch that is excellent in both percutaneous absorbability of a drug and adhesiveness to the skin. An adhesive patch of the present invention includes a backing and an adhesive layer integrally laminated on one side of the backing, the adhesive layer including a drug, levulinic acid, and an acrylic adhesive including an acrylic polymer (A) containing a vinyl-based monomer (I) moiety having a solubility parameter of 9 (cal/cm³)^{1/2} or more. According to the present invention, it is possible to provide an adhesive patch that is excellent in both percutaneous absorbability of a drug and adhesiveness to the skin.

### TECHNICAL FIELD

The present invention relates to an adhesive patch that is excellent in both percutaneous absorbability of a drug and adhesiveness to the skin.

### BACKGROUND ART

Conventionally, a drug has been administered to the human body through the mouth in the form of an oral preparation such as a tablet, a capsule, or a syrup. However, an oral preparation may cause problems such as degradation due to the first-pass effect in the liver, occurrence of gastrointestinal disturbance, or occurrence of a side effect due to a rapid change in the blood concentration.

Thus, there has been proposed a method for administering a drug to the human body through the skin by sticking an adhesive patch, containing a drug, to the skin. The adhesive patch generally includes a backing and an adhesive layer containing an adhesive polymer and a drug. The adhesive patch allows the drug to be gradually absorbed through the skin, allowing for reduction of a side effect caused by an excessive increase in the blood drug concentration. Furthermore, when an intolerable side effect occurs, the transdermal administration of the drug can be immediately stopped by removing the adhesive patch from the skin. Furthermore, for patients who have difficulty swallowing the drug, the drug can be administered easily by simply sticking the adhesive patch.

On the other hand, the skin functions as a barrier to prevent chemical substances and bacteria from entering the body through the skin. Thus, the conventional adhesive patch encounters a problem where insufficient percutaneous absorbability of the drug causes difficulties in achieving the expected percutaneous absorption amount of a drug. In order to solve such a problem, various studies have been conducted on the adhesive patch.

In order to improve the percutaneous absorbability of the drug, it is necessary to keep the drug included in the adhesive layer in a dissolved state and transfer the drug in this dissolved state to the skin surface. However, the drug easily crystallizes in the adhesive layer, which causes difficulties in stably keeping the drug in a dissolved state in the adhesive layer.

Regarding this, Patent Literature 1 discloses an adhesive patch including an adhesive layer containing buprenorphine, which is a type of drug, a carboxylic acid, and a polysiloxane adhesive. This adhesive layer has a structure in which a solution in which buprenorphine is dissolved in the carboxylic acid is dispersed as a droplet in the polysiloxane adhesive. Specific examples of the carboxylic acid include oleic acid, levulinic acid, linoleic acid, and linolenic acid.

### CITATION LIST

### Patent Literature

Patent Literature 1: JP2010-510259A

### SUMMARY OF INVENTION

### Technical Problem

However, the adhesive patch of Patent literature 1 has a problem where using the carboxylic acid causes insufficient cohesive force of the adhesive layer, which leads to low adhesiveness to the skin. As described above, it has been difficult for the conventional adhesive patch to simultaneously achieve percutaneous absorbability of the drug and adhesiveness to the skin.

Accordingly, it is an object of the present invention to provide an adhesive patch that is excellent in both percutaneous absorbability of a drug and adhesiveness to the skin.

### Solution to Problem

An adhesive patch of the present invention includes a backing and an adhesive layer integrally laminated on one side of the backing, the adhesive layer including a drug, levulinic acid, and an acrylic adhesive including an acrylic polymer (A) containing a vinyl-based monomer (I) moiety having a solubility parameter of 9 (cal/cm³)^{1/2} or more.

That is, an adhesive patch of the present invention includes:
a backing; and
an adhesive layer integrally laminated on one side of the backing, the adhesive layer including a drug, levulinic acid, and an acrylic adhesive including an acrylic polymer (A) containing a vinyl-based monomer (I) moiety having a solubility parameter of 9 (cal/cm³)^{1/2} or more.

[Adhesive Layer]

The adhesive patch includes an adhesive layer integrally laminated on one side of the backing. The adhesive layer includes a drug, levulinic acid, and an acrylic adhesive.

(Drug)

The adhesive layer includes a drug. A drug that can be dissolved in levulinic acid is preferable. For example, it is preferable that 0.1 g or more of the drug can be dissolved in 1 mL of levulinic acid at a liquid temperature of 35° C.

Specifically, the solubility of the drug is determined as follows. One mL of levulinic acid is put into a test tube and kept at 35° C. to obtain liquid levulinic acid. To the liquid levulinic acid, 0.1 g of the drug is added to obtain a mixed liquid. While keeping the mixed liquid at 35° C., the mixed liquid in the test tube is vibrated for 10 minutes using an ultrasonic cleaner (e.g., a glasses cleaner). After removing the test tube from the ultrasonic cleaner and leaving it in an environment of 35° C. for 4 hours, the mixed liquid in the test tube is visually observed to check for the presence of residues or precipitates of the drug. In a case where no drug residues or precipitates are observed in the mixed liquid, it is determined that 0.1 g or more of the drug can be dissolved in 1 mL of levulinic acid at a liquid temperature of 35° C. Furthermore, in a case where drug residues or precipitates are observed in the mixed liquid, it is determined that 0.1 g or more of the drug cannot be dissolved in 1 mL of levulinic acid at a liquid temperature of 35° C.

Examples of the drug include an acidic drug, a basic drug, and an amphoteric drug, with a basic drug being preferred. The acidic drug can become an anion (negative ion) by releasing a proton in the adhesive layer. The acidic drug preferably has a molecular structure that releases a proton and becomes an anion (negative ion) in the adhesive layer. Examples of the acidic drug include a drug having an acidic functional group (anionic functional group) such as a carboxyl group (—COOH), a sulfonate group (—SO₃H), or a phosphate group (H₂PO₄⁻) represented by the following chemical formula (1). The basic drug can become a cation (positive ion) by accepting a proton in the adhesive layer. The basic drug preferably has a molecular structure that accepts a proton and becomes a cation (positive ion) in the adhesive layer. Examples of the basic drug include a drug having an amine structure. The amphoteric drug can become both a cation and an anion by releasing and accepting a proton in the adhesive layer. Examples of the amphoteric drug include a compound having both an acidic functional group and the amine structure.

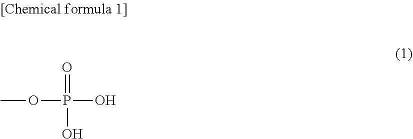

Between the molecular structure that releases a proton and becomes an anion (negative ion) in the adhesive layer and the molecular structure that accepts a proton and becomes a cation (positive ion) in the adhesive layer, the acidic drug preferably has only the molecular structure that releases a proton and becomes an anion (negative ion) in the adhesive layer. That is, the acidic drug preferably does not have the molecular structure that accepts a proton and becomes a cation (positive ion) in the adhesive layer.

Between the molecular structure that releases a proton and becomes an anion (negative ion) in the adhesive layer and the molecular structure that accepts a proton and becomes a cation (positive ion) in the adhesive layer, the basic drug preferably has only the molecular structure that accepts a proton and becomes a cation (positive ion) in the adhesive layer. That is, the basic drug preferably does not have the molecular structure that releases a proton and becomes an anion (negative ion) in the adhesive layer.

The amphoteric drug preferably has both the molecular structure that releases a proton and becomes an anion (negative ion) in the adhesive layer and the molecular structure that accepts a proton and becomes a cation (positive ion) in the adhesive layer.

Examples of the molecular structure that releases a proton and becomes an anion (negative ion) in the adhesive layer include an acidic functional group (anionic functional group) such as a carboxyl group (—COOH), a sulfonate group (—SO₃H), or a phosphate group (H₂PO₄⁻) represented by the above-described chemical formula (1). Examples of the molecular structure that accepts a proton and becomes a cation (positive ion) in the adhesive layer include an amine structure. Examples of the amine structure include the same structures as the following amine structures in the basic drugs.

Examples of the basic drug include a drug having an amine structure. Examples of the amine structure include a primary amine structure, a secondary amine structure, and a tertiary amine structure. A primary amine structure means a structure represented by —NH₂. A secondary amine structure means a structure represented by —NHR¹ (where R¹ is a monovalent organic group bonded to N). A tertiary amine structure means a structure represented by —NR²R³. A tertiary amine structure means a structure represented by the following chemical formula (A). R² and R³ are each independently bonded to N. R² and R³ may be a monovalent organic group. R² and R³ may be bonded to each other directly or by intermediary of one or more atoms. In this case, R² and R³ form a cyclic structure with a nitrogen atom. Note that an organic group means an atomic group containing at least a carbon atom.

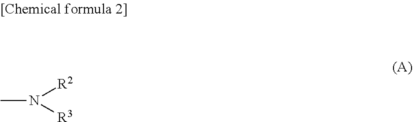

The number of ring members in the above-mentioned cyclic structure is not particularly limited. However, the number is preferably 4 to 20, more preferably 5 to 8. Note that the number of ring members means the number of atoms constituting a basic ring in the cyclic structure. Furthermore, a basic ring means a skeletal ring excluding a substituent in the cyclic structure. In the cyclic structure, the atom constituting the basic ring may include a heteroatom such as an oxygen atom, a sulfur atom, or a nitrogen atom.

Examples of the acidic drug include salicylic acid, aspirin, salsalate, alclofenac, suprofen, ibuprofen, naproxen, flurbiprofen, ketoprofen, fenbufen, glycyrrhetinic acid, sulindac, diflunisal, and tiaprofenic acid.

Examples of the amphoteric drug include tolfenamic acid, mefenamic acid, flufenamic acid, indomethacin, acemetacin, metiazinic acid, protizinic acid, and pranoprofen.

Examples of the drug include blonanserin, buprenorphine, rotigotine, and guanfacine. Among these, blonanserin, rotigotine, and guanfacine are preferable. These drugs, which include an amine structure, are the basic drugs. Thus, their solubility in levulinic acid is high, making it possible to improve percutaneous absorbability of the drug. The drugs may be used singly or in combination of two or more.

For blonanserin, buprenorphine, rotigotine, and guanfacine, 0.1 g or more of each can be dissolved in 1 mL of levulinic acid at a liquid temperature of 35° C.

The drug includes a free form drug and its physiologically acceptable salt. The adhesive layer may include at least one of a free form drug or a salt form drug. Between them, a free form drug is preferable. The free form drug can be used to provide an adhesive patch with better percutaneous absorbability of the drug. Examples of the free form drug include a free base form drug in a case where the drug is a basic drug and a free acid form drug in a case where the drug is an acidic drug.

The basic drug includes a free base form drug and its physiologically acceptable acid addition salt. The physiologically acceptable acid addition salt is not particularly limited. However, examples thereof include an inorganic acid salt such as a hydrochloride, a hydrobromide, a nitrate, a sulfate, and a phosphate; and an organic acid salt such as a formate, an acetate, a trifluoroacetate, an ascorbate, a benzoate, a cinnamate, a citrate, a fumarate, a glutamate, a tartrate, an oxalate, a glutarate, a camphorate, an adipate, a sorbate, a lactate, a maleate, a linoleate, a linolenate, a malate, a malonate, a mandelate, a methanesulfonate (mesylate), a phthalate, a salicylate, a stearate, an isostearate, a succinate, a propionate, a butyrate, a pamoate, a p-toluenesulfonate (tosylate), and a benzenesulfonate (besylate). In particular, a free base form drug is preferable as the basic drug. The free base form drug can provide an adhesive patch with better percutaneous absorbability of the drug.

The content ratio of the drug in the adhesive layer is preferably 0.1 parts by mass or more, more preferably 0.5 parts by mass or more, still more preferably 1 part by mass or more, still more preferably 5 part by mass or more, and particularly preferably 10 parts by mass or more, in 100 parts by mass of the total amount of the drug, levulinic acid, and the acrylic adhesive. Furthermore, the content ratio of the drug in the adhesive layer is preferably 50 parts by mass or less, more preferably 40 parts by mass or less, still more preferably 35 part by mass or less, still more preferably 30 part by mass or less, and still more preferably 25 parts by mass or less, in 100 parts by mass of the total amount of the drug, levulinic acid, and the acrylic adhesive. The content ratio of the drug of 0.1 parts by mass or more can quickly increase the drug blood concentration to a desired range. The content ratio of the drug of 50 parts by mass or less can reduce excessive precipitates of the excessive amount of the drug as crystals in the adhesive layer and improve percutaneous absorbability of the drug and adhesiveness of the adhesive layer.

In particular, the adhesive patch of the present invention, which exhibits the excellent percutaneous absorbability of the drug, can achieve a therapeutically effective blood concentration of the drug even if the content of the drug in the adhesive layer is low. From this point of view, the content ratio of the drug in the adhesive layer is preferably 35 parts by mass or less, more preferably 15 parts by mass or less, still more preferably 10 parts by mass or less, and particularly preferably 8 parts by mass or less, in 100 parts by mass of the total amount of the drug, levulinic acid, and the acrylic adhesive.

Note that when the content ratio of each constituent component included in the adhesive layer and the mass proportion (e.g., mass ratio, etc.) of the constituent components included in the adhesive layer are calculated in the adhesive patch of the present invention, in a case of using a salt of the drug instead of a free form drug as the drug, the mass of the drug in terms of the free form drug converted from the salt of the drug is used as the mass of the salt of the drug. The mass of the drug in terms of the free form drug converted from the salt of the drug is defined as the mass of the free form drug in an equivalent molar amount of the salt of the drug.

(Levulinic Acid)

The adhesive layer includes levulinic acid. The drug can be satisfactorily dissolved in levulinic acid. Thus, using levulinic acid allows the drugs to be included in the adhesive layer in a dissolved state. The drug dissolved by levulinic acid can easily diffuse and move in the adhesive layer and has excellent skin permeability. Thus, using levulinic acid allows for the provision of the adhesive patch with excellent percutaneous absorbability of the drug.

The basic drug is preferably used as the drug. The basic drugs can accept a proton and become a cation in the adhesive layer. On the other hand, levulinic acid has a carboxyl group, and the carboxyl group contained in levulinic acid is ionized and becomes an anion (—COO—) in the adhesive layer. The basic drug accepts a proton and becomes a cation, while the carboxyl group contained in levulinic acid is ionized and generates an anion. As a result, the cation derived from the basic drug and the anion derived from levulinic acid form an ionic bond, which further improves the lipid solubility of the basic drug and makes it easier for the basic drug to permeate the skin. Thus, it becomes possible to further improve the percutaneous absorbability of the basic drug.

According to the present invention, the percutaneous absorbability of the drug can be improved, so that the content of the drug in the adhesive patch can be reduced. This can reduce the occurrence of an unexpected side effect due to excessive drug absorption into the body. Furthermore, this can reduce the application area of the adhesive patch, which may reduce discomfort felt by the patient while the adhesive patch is applied, and also to reduce repeated application of the adhesive patch to the same site when the adhesive patch is replaced.

The content ratio of levulinic acid in the adhesive layer is preferably 1 part by mass or more, more preferably 2 parts by mass or more, still more preferably 3 parts by mass or more, and particularly preferably 5 parts by mass or more, in 100 parts by mass of the total amount of the drug, levulinic acid, and the acrylic adhesive. The content ratio of levulinic acid in the adhesive layer is preferably 25 parts by mass or less, more preferably 20 parts by mass or less, and particularly preferably 15 parts by mass or less, in 100 parts by mass of the total amount of the drug, levulinic acid, and the acrylic adhesive. Setting the content ratio of levulinic acid to 1 part by mass or more makes it possible to efficiently dissolve a sufficient amount of the drug in the adhesive layer, and improve percutaneous absorbability of the drug. Setting the content ratio of levulinic acid to 25 parts by mass or less makes it possible to reduce leakage (bleeding) of an excessive amount of levulinic acid to the surface of the adhesive layer, thereby maintaining the excellent adhesiveness of the adhesive patch.

The mass ratio of levulinic acid to the drug [(mass of levulinic acid)/(mass of drug)] in the adhesive layer is preferably 0.2 or more, more preferably 0.3 or more, and particularly preferably 0.4 or more. On the other hand, the mass ratio of levulinic acid to the drug [(mass of levulinic acid)/(mass of drug)] in the adhesive layer is preferably 4 or less, more preferably 3 or less, still more preferably 2.5 or less, still more preferably 1.5 or less, and still more preferably 1.0 or less. Setting the above-described mass ratio to 0.2 or more makes it possible to efficiently dissolve a sufficient amount of the drug in the adhesive layer, and improve percutaneous absorbability of the drug. Setting the above-described mass ratio to 4 or less makes it possible to prevent the suppression of the diffusion migration of the drug due to an excessive amount of levulinic acid in the adhesive layer, and also to stably release the drug from the adhesive patch.

(Acrylic Adhesive)

The adhesive layer includes an acrylic adhesive. The acrylic adhesive includes an acrylic polymer (A) containing a vinyl-based monomer (I) moiety having a solubility parameter of 9 (cal/cm³)^{1/2} or more.

The vinyl-based monomer means a monomer having an ethylenically unsaturated double bond.

In the present invention, using levulinic acid allows the drug to exist in a dissolved state in the adhesive layer. However, levulinic acid having low affinity with an adhesive may leak excessively to the surface of the adhesive layer and reduce the adhesiveness of the adhesive layer. Thus, it may be difficult to simultaneously achieve percutaneous absorbability of the drug and adhesiveness in the adhesive layer using levulinic acid. However, the acrylic polymer (A) containing the vinyl-based monomer (I) moiety having a solubility parameter of 9 (cal/cm³)^{1/2} or more appropriately improves affinity for levulinic acid. Using this acrylic polymer (A) as an acrylic adhesive can appropriately keep levulinic acid in the adhesive layer and reduce excessive leakage of levulinic acid to the surface of the adhesive layer. As a result, the adhesive layer can exhibit the excellent adhesiveness. Thus, according to the present invention, using a combination of levulinic acid and the acrylic polymer (A) allows for the provision of the adhesive patch that simultaneously achieves percutaneous absorbability of the drug and adhesiveness.

The drug exists in a dissolved state in the adhesive layer. However, a part of the drug may precipitate as crystals. A storage temperature may change during storage of the adhesive patch. It is preferable that, even if the storage temperature of the adhesive patch changes, the dissolution state and precipitation state of the drug remain unchanged in the adhesive layer before and after storage. This is because a change in the dissolution state and precipitation state of the drug may change the percutaneous absorbability of the drug, so that an intended medicinal effect may not be obtained, thereby affecting a treatment. In particular, if crystals of the drug precipitate excessively due to a change in the storage temperature of the adhesive patch, the percutaneous absorbability of the drug may become inconsistent or deteriorate.

In the present invention, as described above, using the acrylic adhesive including the acrylic polymer (A) makes it possible to appropriately keep levulinic acid in the adhesive layer. As a result, the dissolved state of the drug can be stably maintained in the adhesive layer. Thus, even if the storage temperature of the adhesive patch changes, change in the dissolution state and precipitation state of the drug is suppressed in the adhesive layer, allowing for reduction of changes in the percutaneous absorbability of the drug. As described above, according to the present invention, it is possible to provide an adhesive patch that is also excellent in drug storage stability.

(Vinyl-Based Monomer (I))

The acrylic polymer (A) contains a moiety derived from a vinyl-based monomer (I) having a solubility parameter of 9 (cal/cm³)^{1/2} or more (also simply referred to as a “vinyl-based monomer (I) moiety”). Specifically, the acrylic polymer (A) is a polymer of monomers including a vinyl-based monomer (I) having a solubility parameter of 9 (cal/cm³)^{1/2} or more. Note that the vinyl-based monomer (I) having a solubility parameter of 9 (cal/cm³)^{1/2} or more may be simply referred to as a “vinyl-based monomer (I)”.

The vinyl-based monomer (I) may or may not have a functional group having an affinity for the drug, but preferably does not have a functional group having an affinity for the drug. This can improve the percutaneous absorbability of the drug. Examples of the functional group having an affinity for the drug in the vinyl-based monomer (I) include the same functional groups as those having an affinity for the drug described later for the acrylic polymer (A).

The solubility parameter of the vinyl-based monomer (I) is 9 (cal/cm³)^{1/2} or more, preferably 9.2 (cal/cm³)^{1/2} or more, more preferably 9.4 (cal/cm³)^{1/2} or more, and particularly preferably 9.5 (cal/cm³)^{1/2} or more. The solubility parameter of the vinyl-based monomer (I) is preferably 15 (cal/cm³)^{1/2} or less, more preferably 14 (cal/cm³)^{1/2} or less, and particularly preferably 11 (cal/cm³)^{1/2} or less. The solubility parameter of the vinyl-based monomer (I) of 9 (cal/cm³)^{1/2} or more makes it possible to improve the affinity of the acrylic polymer (A) for levulinic acid. As a result, levulinic acid can be appropriately retained in the adhesive layer, and excessive leakage of levulinic acid to the surface of the adhesive layer can be reduced to maintain excellent adhesiveness of the adhesive layer. The solubility parameter of the vinyl-based monomer (I) of 15 (cal/cm³) 2 or less makes it possible to prevent the affinity between the acrylic polymer (A) and levulinic acid from becoming too high. Accordingly, the adhesive patch can gradually release levulinic acid from the adhesive layer after application, and therefore, can also gradually release the drug from the adhesive layer. As a result, the percutaneous absorbability of the drug can be further improved while maintaining excellent adhesiveness.

Examples of the vinyl-based monomer (I) include N-vinyl-2-pyrrolidone (SP value: 9.7 (cal/cm³)^{1/2}), diacetone acrylamide (SP value: 9.8 (cal/cm³)^{1/2}), methyl acrylate (SP value: 9.4 (cal/cm³)^{1/2}), 2-hydroxyethyl acrylate (SP value: 12.1 (cal/cm³)^{1/2}), 2-hydroxyethyl methacrylate (SP value: 11.3 (cal/cm³)^{1/2}), and acrylic acid (11.7 (cal/cm³)^{1/2}). As the vinyl-based monomer (I), at least one selected from the group consisting of N-vinyl-2-pyrrolidone, diacetone acrylamide, methyl acrylate, and 2-hydroxyethyl acrylate is preferably contained. Note that the numerical values in parentheses are solubility parameters of respective vinyl-based monomer (I). Among these, the vinyl-based monomer (I) is preferably N-vinyl-2-pyrrolidone, methyl acrylate, or 2-hydroxyethyl acrylate, with N-vinyl-2-pyrrolidone being more preferred. The vinyl-based monomer (I) preferably contains N-vinyl-2-pyrrolidone. The vinyl-based monomer (I) preferably contains methyl acrylate and 2-hydroxyethyl acrylate. These make it possible to provide an adhesive patch having better percutaneous absorbability of a drug and adhesiveness. The vinyl-based monomer (I) may be used singly or in combination of two or more thereof.

Methods for defining and calculating the solubility parameter are described in the literature “Hansen Solubility Parameters: A User's Handbook” (Charles M. Hansen, CRC Press, Jun. 15, 2007″, pages 28 to 30).

The values described in the above-mentioned literature “Hansen Solubility Parameters: A User's Handbook” can be used as the solubility parameters of vinyl-based monomers such as the vinyl-based monomer (I) and a vinyl-based monomer (II), to be described later. In addition, for vinyl-based monomers for which the solubility parameters are not described in the above-mentioned literature, the solubility parameters can be calculated on the basis of the following Equation (i) proposed by Hansen:

δ=(δ_{d}²+δₚ²+δₕ²)^{1/2} (i)

(In Equation (i), δ is a solubility parameter, δ_{d} a term (dispersion term) according to the dispersive force (van der Waals' force) of London, δₚ a term (polar term) according to the polarity of the molecule, and δₕ a term (hydrogen bond term) according to the hydrogen bond).

δ_{d}, δₚ and δₕ in the above-described Equation (i) can be obtained by the following Equations (ii) to (iv) based on the Van Krevelen and Hoftyzer method:

δ_{d}=ΣF_{di}/V (ii)

δₚ=(ΣFₚᵢ²)^{1/2}/V (iii), and

δₕ=(ΣEₕᵢ/V)^{1/2} (iv)

(F_{di} in Equation (ii) is the molar attraction constant due to the dispersive force of London, Fₚᵢ in Equation (iii) is the molar attraction constant due to the dipole-dipole force, Eₕᵢ in Equation (iv) is the hydrogen bond energy, and V in Equations (ii) to (iv) is the molar volume of the vinyl-based monomer).

In the present invention, the molar attraction constants F_{di}, Fₚᵢ and Eₕᵢ are determined by the Van Krevelen and Hoftyzer method. The molar volume V is a value calculated by dividing the molar mass of the vinyl-based monomer by the density of the vinyl-based monomer.

The content of the vinyl-based monomer (I) moiety in the acrylic polymer (A) is preferably 5% by mass or more, more preferably 7% by mass or more, still more preferably 10% by mass or more, and particularly preferably 15% by mass or more. The content of the vinyl-based monomer (I) moiety in the acrylic polymer (A) is preferably 40% by mass or less, more preferably 35% by mass or less, still more preferably 30% by mass or less, and particularly preferably 27% by mass or less. Setting the content of the vinyl-based monomer (I) moiety to 5% by mass or more makes it possible to improve the affinity of the acrylic polymer (A) for levulinic acid, whereby levulinic acid can be appropriately retained in the adhesive layer, and excessive leakage of levulinic acid to the surface of the adhesive layer can be reduced. On the other hand, setting the content of the vinyl-based monomer (I) moiety to 35% by mass or less makes it possible to appropriately maintain the cohesive force of the adhesive layer and the excellent adhesiveness of the adhesive layer.

(Vinyl-Based Monomer (II))

The acrylic polymer (A) preferably contains a moiety derived from a vinyl-based monomer (II) having a solubility parameter of less than 9 (cal/cm³)^{1/2} (also simply referred to as a “vinyl-based monomer (II) moiety”). Specifically, the acrylic polymer (A) is preferably a copolymer of monomers including the vinyl-based monomer (I) having a solubility parameter of 9 (cal/cm³)^{1/2} or more and the vinyl-based monomer (II) having a solubility parameter of less than 9 (cal/cm³)^{1/2}. Using the vinyl-based monomer (II) makes it possible to impart a more excellent adhesive force to the adhesive layer. Note that the vinyl-based monomer (II) having a solubility parameter of less than 9 (cal/cm³)^{1/2} may be simply referred to as a “vinyl-based monomer (II)”.

The vinyl-based monomer (II) may or may not contain a functional group having an affinity for the drug, but preferably does not contain a functional group having an affinity for the drug. This can impart a more excellent adhesive force to the adhesive layer while suppressing a decrease in the percutaneous absorbability of the drug due to the functional group having an affinity for the drug. Examples of the functional group having an affinity for the drug in the vinyl-based monomer (II) include the same functional groups as those having an affinity for the drug described later for the acrylic polymer (A).

The solubility parameter of the vinyl-based monomer (II) is preferably less than 9 (cal/cm³)^{1/2}, more preferably 8.8 (cal/cm³)^{1/2} or less, and particularly preferably 8.6 (cal/cm³)^{1/2} or less. The solubility parameter of the vinyl-based monomer (II) is preferably 7 (cal/cm³)^{1/2} or more, and more preferably 7.5 (cal/cm³) or more. Using the vinyl-based monomer (II) having a solubility parameter of less than 9 (cal/cm³) 172 makes it possible to adjust the affinity between levulinic acid and the acrylic polymer (A) in the adhesive layer to an appropriate level. Accordingly, the adhesive patch can gradually release levulinic acid from the adhesive layer after application, and therefore, can also gradually release the drug from the adhesive layer. As a result, the percutaneous absorbability of the drug can be further improved while maintaining excellent adhesiveness. The solubility parameter of the vinyl-based monomer (II) of 7 (cal/cm³)^{1/2} or more makes it possible to improve the affinity between levulinic acid and the acrylic polymer (A), thereby reducing excessive leakage of levulinic acid from the surface of the adhesive layer to improve the adhesiveness to the skin.

Examples of the vinyl-based monomer (II) include an alkyl(meth)acrylate, vinyl acetate, vinyl chloride, an α-olefin, and styrene. Examples of the α-olefin include ethylene, propylene, 1-butene, 1-pentene, 1-hexene, 4-methyl-1-pentene, 1-octene, 1-nonene, 1-decene, 1-tetradecene, 1-hexadecene, 1-octadecene, and 1-eicosene. Among these, the vinyl-based monomer (II) is preferably an alkyl(meth)acrylate. The alkyl (meth)acrylate can impart an appropriate cohesive force to the adhesive layer to improve the adhesiveness of the adhesive layer. The vinyl-based monomer (II) may be used singly or in combination of two or more thereof. The term “(meth)acrylate” means acrylate or methacrylate.

The alkyl group of the alkyl (meth)acrylate is a group represented by —CₙH₂ₙ₊₁ (wherein n is a positive integer). The alkyl group of the alkyl (meth)acrylate preferably has 1 to 16 carbon atoms, more preferably 1 to 14 carbon atoms, still more preferably 2 to 12 carbon atoms, and particularly preferably 2 to 10 carbon atoms.

Examples of the alkyl (meth)acrylate include ethyl acrylate (SP value: 8.8 (cal/cm³)^{1/2}), n-propyl acrylate (SP value: 8.7 (cal/cm³)^{1/2}), isopropyl acrylate (SP value: 8.5 (cal/cm³)^{1/2}) n-butyl acrylate (SP value: 8.6 (cal/cm³)^{1/2}), isobutyl acrylate (SP value: 8.5 (cal/cm³)^{1/2}), hexyl acrylate (SP value: 8.4 (cal/cm³)^{1/2}), n-octyl acrylate (SP value: 8.2 (cal/cm³)^{1/2}), isooctyl acrylate (SP value: 8.2 (cal/cm³) 372), 2-ethylhexyl acrylate (SP value: 7.8 (cal/cm³)^{1/2}), decyl acrylate (SP value: 8.3 (cal/cm³)^{1/2}), dodecyl acrylate (SP value: 8.3 (cal/cm³)^{1/2}), tridecyl acrylate (SP value: 8.3 (cal/cm³)^{1/2}), hexadecyl acrylate (SP value: 8.2 (cal/cm³)^{1/2}) and cyclohexyl acrylate (SP value: 8.8 (cal/cm³)^{1/2}); and methyl methacrylate (SP value: 8.8 (cal/cm³)^{1/2}), n-propyl methacrylate (SP value: 8.8 (cal/cm³)^{1/2}), isopropyl methacrylate (SP value: 8.3 (cal/cm³)^{1/2}), n-butyl methacrylate (SP value: 8.9 (cal/cm³)^{1/2}), isobutyl methacrylate (SP value: 8.3 (cal/cm³)^{1/2}), hexyl methacrylate (SP value: 8.2 (cal/cm³)^{1/2}), n-octyl methacrylate (SP value: 8.4 (cal/cm³)^{1/2}), 2-ethylhexyl methacrylate (SP value: 8.3 (cal/cm³)^{1/2}), decyl methacrylate (SP value: 8.3 (cal/cm³)^{1/2}\), dodecyl methacrylate (SP value: 8.2 (cal/cm³)^{1/2}), tridecyl methacrylate (SP value: 8.3 (cal/cm³)^{1/2}) and cyclohexyl methacrylate (SP value: 8.8 (cal/cm³)^{1/2}). The numerical values in parentheses are solubility parameters of respective alkyl (meth)acrylates. The alkyl (meth)acrylate may be used singly or in combination of two or more.

Among these, the alkyl (meth)acrylate is preferably ethyl acrylate, 2-ethylhexyl acrylate, 2-ethylhexyl methacrylate, n-octyl acrylate, or dodecyl methacrylate.

The alkyl (meth)acrylate preferably contains at least one selected from the group consisting of ethyl acrylate, 2-ethylhexyl acrylate, 2-ethylhexyl methacrylate, n-octyl acrylate, and dodecyl methacrylate.

The vinyl-based monomer (II) preferably includes at least one of 2-ethylhexyl acrylate and 2-ethylhexyl methacrylate. The vinyl-based monomer (II) preferably includes n-octyl acrylate and ethyl acrylate.

The content of the alkyl (meth)acrylate in the vinyl-based monomer (II) is preferably 80% by mass or more, more preferably 90% by mass or more, and particularly preferably 100% by mass. In other words, it is particularly preferable that the vinyl-based monomer (II) consist only of the alkyl(meth)acrylate.

The content of the vinyl-based monomer (II) moiety in the acrylic polymer (A) is preferably 95% by mass or less, more preferably 93% by mass or less, still more preferably 90% by mass or less, and particularly preferably 85% by mass or less. The content of the vinyl-based monomer (II) moiety in the acrylic polymer (A) is preferably 60% by mass or more, more preferably 65% by mass or more, still more preferably 70% by mass or more, still more preferably 73% by mass or more, and particularly preferably 75% by mass or more. Setting the content of the vinyl-based monomer (II) moiety to 95% by mass or less allows the vinyl-based monomer (I) moiety described above to be contained in the acrylic polymer (A) in a sufficient amount. Setting the content of the vinyl-based monomer (II) moiety to 60% by mass or more allows the adhesive layer to have appropriate cohesive force, thereby improving the adhesiveness of the adhesive layer.

The acrylic polymer (A) is preferably a copolymer of monomers including the vinyl-based monomer (I) having a solubility parameter of 9 (cal/cm³)^{1/2} or more and the vinyl-based monomer (II) having a solubility parameter of less than 9 (cal/cm³)^{1/2}. The total content of the vinyl-based monomer (I) moiety and the vinyl-based monomer (II) moiety in the above-mentioned copolymer is preferably 70% by mass or more, preferably 80% by mass or more, more preferably 90% by mass or more, still more preferably 95% by mass or more, still more preferably 99% by mass or more, and particularly preferably 100% by mass. Setting the total content of the vinyl-based monomer (I) moiety and the vinyl-based monomer (II) moiety to 70% by mass or more makes it possible to give an adhesive patch excellent in both percutaneous absorbability of a drug and adhesiveness.

Incidentally, the acrylic polymer (A) preferably does not have a functional group having an affinity for the drug from the viewpoint of percutaneous absorption.

A functional group having an affinity for the drug is a functional group capable of ionizing into an anion or a cation in the adhesive layer to form an ionic bond with the drug. When the acrylic polymer (A) has a functional group having an affinity for the drug, the affinity of the functional group makes it difficult for the drug to diffuse and migrate in the adhesive layer, so that the drug remains in the adhesive layer. This phenomenon may reduce the percutaneous absorbability of the drug. Therefore, in the present invention, it is preferable that the acrylic polymer (A) have no functional group having an affinity for the drug. That is, in the acrylic polymer (A), the content of the monomer moiety containing a functional group having an affinity for the drug is particularly preferably 0% by mass.

The acrylic polymer (A) containing no functional group having an affinity for the drug can further improve percutaneous absorbability of the drug without suppression of diffusion migration of the drug in the adhesive layer.

As described above, from the viewpoint of percutaneous absorption, it is preferable that the acrylic polymer (A) have no functional group having an affinity for the drug. From the viewpoint of suppressing the volatilization and decomposition of the drug during the manufacture or storage of the adhesive patch, however, the acrylic polymer (A) having a functional group having an affinity for the drug may be selected.

The content of the monomer moiety containing a functional group having an affinity for the drug in the acrylic polymer (A) can be appropriately determined according to the type of the drug, and the manufacturing conditions and storage conditions of the adhesive patch. However, in the case where the acrylic polymer (A) has a functional group having an affinity for the drug, the content of the monomer moiety containing a functional group having an affinity for the drug in the acrylic polymer (A) is preferably 10% by mass or less, more preferably 5% by mass or less, still more preferably 1% by mass or less, still more preferably 0.1% by mass or less, and particularly preferably 0.05% by mass or less, from the viewpoint of balance with the percutaneous absorbability, which is the basic required performance as an adhesive patch.

Even when the acrylic polymer (A) has a functional group having an affinity for the drug, setting the content of the monomer moiety containing a functional group having an affinity for the drug to 10% by mass or less makes it possible to prevent the suppression of the diffusion migration of the drug in the adhesive layer due to the affinity of the functional group, and to improve the percutaneous absorbability of the drug.

Specific examples of the functional group having an affinity for the drug include an anionic functional group such as a carboxyl group (—COOH), a sulfonate group, and a phosphate group, and a cationic functional group such as an amino group (—NH₂), and a monosubstituted amino group in which one of the hydrogen atoms of the amino group is substituted with another atom or a monovalent organic group.

Examples of the drug include an acidic drug, a basic drug, and a neutral drug. These drugs become a cation or an anion by accepting or releasing protons (H⁺) in the adhesive layer. On the other hand, if the acrylic polymer (A) has a functional group having an affinity for the drug, the functional group having an affinity for the drug is ionized in the adhesive layer, so that the residue moiety of the functional group remaining bonded to the polymer chain of the acrylic polymer (A) becomes an anion or a cation. Then, an ionic bond is generated between the cation or anion moiety of the drug and the residue moiety of the functional group of the acrylic polymer (A), and as a result, the acrylic polymer (A) traps the drug, so that the drug hardly diffuses and migrates in the adhesive layer.

For example, when the acrylic polymer (A) has an anionic functional group such as a carboxyl group, a sulfonate group, or a phosphate group as a functional group having affinity for the drug, these anionic functional groups generate —COO⁻, —SO₃⁻ or a structure (—OPO₃²⁻) represented by the following chemical formula (2) as a residue moiety which remains bonded to the polymer chains of the acrylic polymer (A) after ionization, and become an anion. In addition, for example, when the acrylic polymer (A) has a cationic functional group such as an amino group or a monosubstituted amino group as a functional group having an affinity for the drug, these cationic functional groups generate —NH₃⁺ or a structure represented by the following chemical formula (3) as a residue moiety which remains bonded to the polymer chains of the acrylic polymer (A) after ionization, or become a cation.

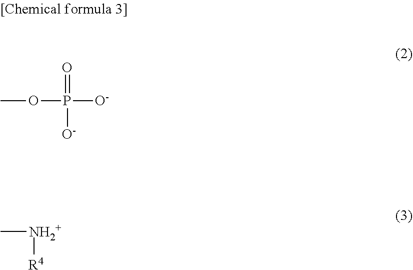

In the chemical formula (3), R⁴ is bonded to a nitrogen atom and is an atom other than a hydrogen atom or a monovalent organic group.

In particular, a basic drug, such as a drug having an amine structure, accepts a proton in the adhesive layer to become a cation. On the other hand, when the acrylic polymer (A) has the above-described anionic functional group as the functional group having an affinity for the drug, the anionic functional group releases a proton to become an anion. As a result, an ionic bond is generated between the cationic moiety of the basic drug and the anionic moiety consisting of the residue moiety of the anionic functional group which remains bonded to the polymer chain of the acrylic polymer (A), so that the acrylic polymer (A) may trap the drug. Thus, the drug hardly diffuses and migrates in the adhesive layer.

In the functional group having an affinity for the drug, the “monosubstituted amino group” refers to a functional group in which one of the hydrogen atoms of the amino group is substituted by another atom or a monovalent organic group. Specifically, the “monosubstituted amino group” is a functional group represented by —NHR⁴ (R⁴ is bonded to a nitrogen atom and is an atom other than a hydrogen atom or a monovalent organic group). Examples of the monosubstituted amino group include a monoalkyl-substituted amino group in which one hydrogen atom of the amino group (—NH₂) is substituted with an alkyl group. An alkyl group is a group represented by —CₙH₂ₙ₊₁ (in the formula, n is a positive integer). Examples of the monoalkyl-substituted amino group include a methylamino group, an ethylamino group, and a propylamino group.

Examples of the monomer containing a functional group having an affinity for the drug include a vinyl-based monomer containing a functional group having an affinity for the drug. Examples of the vinyl-based monomer containing a functional group having an affinity for the drug include:
a carboxyl group-containing vinyl-based monomer such as acrylic acid, methacrylic acid, itaconic acid, maleic acid, mesaconic acid, citraconic acid, glutaconic acid, and butylmaleic acid;
a sulfonate group-containing (meth)acrylate such as 3-sulfopropyl (meth)acrylate;
a phosphate group-containing (meth)acrylate such as 2-acryloyloxyethyl acid phosphate and 2-methacryloyloxyethyl acid phosphate; and
an amino group- or monoalkyl-substituted amino group-containing (meth)acrylate such as aminoethyl (meth)acrylate, ethylaminoethyl (meth)acrylate, aminopropyl (meth)acrylate, ethylaminopropyl (meth)acrylate, and acrylamide.

The acrylic polymer (A) may be formed by a conventionally known method. Example thereof include a method of polymerizing the above-described monomers in the presence of a polymerization initiator. Specifically, a predetermined amount of monomers, a polymerization initiator, and a polymerization solvent are fed to a reaction vessel and heated at a temperature of 60 to 80° C. for 4 to 48 hours to subject the monomers to a radical polymerization.

Examples of the polymerization initiator include an azobis-based polymerization initiator such as 2,2′-azobisisobutyronitrile (AIBN), 1,1′-azobis(cyclohexane-1-carbonitrile), and 2,2′-azobis-(2,4′-dimethylvaleronitrile); and a peroxide-based polymerization initiator such as benzoyl peroxide (BPO), lauroyl peroxide (LPO), and di-tert-butylperoxide. Examples of the polymerization solvent include ethyl acetate, cyclohexane, and toluene. Furthermore, the polymerization reaction is preferably carried out under a nitrogen gas atmosphere.

The acrylic polymer (A) may be crosslinked. Examples of the crosslinking method of the acrylic polymer (A) include a chemical crosslinking method using an organic peroxide, a crosslinking aid, a crosslinking agent, and the like, and a physical crosslinking method of irradiating ionizing radiation. Examples of the ionizing radiation include electron beams, α-rays, β-rays, and γ-rays.

Examples of the organic peroxide include benzoyl peroxide, acetyl peroxide, decanoyl peroxide, lauroyl peroxide, dicumyl peroxide, di-t-butyl peroxide, t-butyl peroxybenzoate, t-butyl peroxy-2-ethylhexanoate, cumene hydroperoxide, and t-butyl hydroperoxide.

Examples of the crosslinking aid include divinylbenzene, trimethylolpropane trimethacrylate, trimethylolpropane triacrylate, 1,6-hexanediol di(meth)acrylate, 1,9-nonanediol di(meth)acrylate, a trimellitic acid triallyl ester, triethylene glycol diacrylate, tetraethylene glycol diacrylate, cyanoethyl acrylate, and bis(4-acryloxypolyethoxyphenyl)propane.

Examples of the crosslinking agent include an isocyanate-based compound (for example, tetramethylene diisocyanate, hexamethylene diisocyanate, isophorone diisocyanate, xylylene diisocyanate, tolylene diisocyanate, 4,4-diphenylmethane diisocyanate, a trimeric adduct of trimethylolpropane and hexamethylene diisocyanate, etc.); an aziridine-based compound (for example, 2,2-bishydroxymethylbutanol-tris[3-(1-aziridinyl)propionic acid], 4,4-bis(ethyleneiminocarbonylamino)diphenylmethane, etc.); an organometallic compound (for example, zirconium and zinc alaninate, zinc acetate, glycine ammonium zinc, a titanium compound, etc.); a metal alcoholate (for example, tetraethyl titanate, tetraisopropyl titanate, aluminum isopropylate, aluminum sec-butyrate, etc.); and a metal chelate compound (for example, dipropoxy bis(acetylacetonate)titanium, tetraoctylene glycol titanium, aluminum isopropylate, ethyl acetoacetate aluminum diisopropylate, aluminum tris(ethyl acetoacetate), aluminum tris(acetylacetonate), etc.).

When the acrylic polymer (A) is crosslinked by a chemical crosslinking method, the acrylic polymer (A) can be crosslinked by heating the acrylic polymer (A) to a temperature at which the crosslinking reaction proceeds in the presence of an organic peroxide, a crosslinking aid, or a crosslinking agent. When a crosslinking aid is used, the acrylic polymer (A) having been crosslinked by the crosslinking aid can also be obtained by supplying, in addition to the monomers of the acrylic polymer (A), a polymerization initiator and a polymerization solvent, a crosslinking aid to a reaction vessel at the time of polymerization of the acrylic polymer (A) and further performing a radical polymerization.

The acrylic polymer (A) may be crosslinked as described above. However, if the acrylic polymer (A) is crosslinked, the cohesive force of the adhesive layer may excessively increase, so that diffusion migration of the drug in the adhesive layer may be suppressed to reduce the percutaneous absorbability of the drug. Therefore, it is preferable that the acrylic polymer (A) have not been crosslinked.

When the acrylic polymer (A) included in the adhesive layer is not crosslinked, the gel fraction of the adhesive layer is preferably 1% by mass or less, more preferably 0.5% by mass or less, still more preferably 0.1% by mass or less, still more preferably 0.05% by mass or less, and particularly preferably 0% by mass.

The gel fraction of the adhesive layer is a value measured in the following manner. The mass [W₀ (g)] of the adhesive layer is measured, and then, the adhesive layer is immersed in xylene at 120° C. for 24 hours. Then, the insoluble portion is filtered through a 200-mesh wire mesh, and the residue on the wire mesh is vacuum-dried. The mass [W₁ (g)] of the obtained dry residue is measured, and the gel fraction of the adhesive layer is calculated by the following formula:

Gel fraction (% by mass)=(W₁/W₀×100.

The content of the acrylic polymer (A) in the acrylic adhesive is preferably 80% by mass or more, more preferably 90% by mass or more, and particularly preferably 100% by mass. That is, it is particularly preferable that the acrylic adhesive consist only of the acrylic polymer (A). Setting the content of the acrylic polymer (A) to 80% by mass or more allows for the provision of an adhesive patch excellent in adhesiveness and percutaneous absorbability of a drug.

The content ratio of the acrylic adhesive in the adhesive layer is preferably 45 parts by mass or more, more preferably 50 parts by mass or more, still more preferably 55 parts by mass or more, still more preferably 60 parts by mass or more, and particularly preferably 70 parts by mass or more, in 100 parts by mass of the total amount of the drug, levulinic acid, and the acrylic adhesive. The content ratio of the acrylic adhesive in the adhesive layer is preferably 95 parts by mass or less, preferably 90 parts by mass or less, more preferably 85 parts by mass or less, and particularly preferably 80 parts by mass or less, in 100 parts by mass of the total amount of the drug, levulinic acid, and the acrylic adhesive. The content ratio of the acrylic adhesive of 45 parts by mass or more makes it possible to improve the adhesiveness of the adhesive layer to the skin. Setting the content ratio of the acrylic adhesive to 95 parts by mass or less makes it possible to add a drug or other additives to the adhesive layer in required amounts.

(Plasticizer)

The adhesive layer preferably includes a plasticizer. The plasticizer can improve the adhesiveness of the adhesive layer. Examples of the plasticizer include esters such as isopropyl myristate, decyl oleate, and isopropyl adipate, monovalent alcohols such as myristyl alcohol, cetanol, octyldodecanol, isostearyl alcohol, and stearyl alcohol, divalent alcohols such as octanediol, and liquid paraffin. Among these, isopropyl myristate, isopropyl adipate, and octyldodecanol are preferred, with isopropyl myristate being more preferred. The plasticizer may be used singly or in combination of two or more kinds thereof.

The content of the plasticizer in the adhesive layer is preferably 1 part by mass or more, more preferably 5 parts by mass or more, and particularly preferably 10 parts by mass or more, relative to 100 parts by mass of the acrylic adhesive. The content of the plasticizer in the adhesive layer is preferably 50 parts by mass or less, and more preferably 45 parts by mass or less, relative to 100 parts by mass of the acrylic adhesive. The content of the plasticizer of 1 part by mass or more can improve the adhesiveness of the adhesive layer by the plasticizer. The content of the plasticizer of 50 parts by mass or less can maintain excellent cohesive force of the adhesive layer.

When the drug included in the adhesive layer contains free base form guanfacine or a physiologically acceptable salt thereof, the content of the plasticizer in the adhesive layer is preferably 1 part by mass or more, more preferably 5 parts by mass or more, and particularly preferably 10 parts by mass or more, relative to 100 parts by mass of the acrylic adhesive. When the drug included in the adhesive layer contains free base form guanfacine or a physiologically acceptable salt thereof, the content of the plasticizer in the adhesive layer is preferably 50 parts by mass or less, more preferably 25 parts by mass or less, and particularly preferably 15 parts by mass or less, relative to 100 parts by mass of the acrylic adhesive. The content of the plasticizer of 1 part by mass or more can improve the adhesiveness of the adhesive layer by the plasticizer. The content of the plasticizer of 50 parts by mass or less can maintain excellent cohesive force of the adhesive layer.

When the drug included in the adhesive layer contains free base form rotigotine or a physiologically acceptable salt thereof, the content of the plasticizer in the adhesive layer is preferably 1 part by mass or more, more preferably 5 parts by mass or more, and even more preferably parts by mass or more, relative to 100 parts by mass of the acrylic adhesive. When the drug included in the adhesive layer contains free base form rotigotine or a physiologically acceptable salt thereof, the content of the plasticizer in the adhesive layer is preferably 50 parts by mass or less, more preferably 45 parts by mass or less, still more preferably 35 parts by mass or less, still more preferably 20 parts by mass or less, and still more preferably 15 parts by mass or less, relative to 100 parts by mass of the acrylic adhesive. The content of the plasticizer of 1 part by mass or more can improve the adhesiveness of the adhesive layer by the plasticizer. The content of the plasticizer of 50 parts by mass or less can maintain excellent cohesive force of the adhesive layer.

The adhesive layer may include other additives such as a tackifier and a filler as long as the adhesive layer does not affect the skin irritation of the adhesive patch.

(Tackifier)

Examples of the tackifier include a terpene resin, a modified terpene resin, a hydrogenated terpene resin, a terpene phenolic resin, rosin, hydrogenated rosin, a rosin ester, a petroleum resin, a coumarone-indene resin, a phenolic resin, a xylene resin, and an alicyclic saturated hydrocarbon resin. The tackifier may be used singly or in combination of two or more. The content of the tackifier in the adhesive layer is preferably 20 to 80 parts by mass, and more preferably 30 to 70 parts by mass, relative to 100 parts by mass of the acrylic adhesive.

(Fillers)

The filler is used to adjust the shape retentivity of the adhesive layer. Examples of the filler include inorganic fillers such as light anhydrous silicic acid, titanium oxide, and zinc oxide; organometallic salts such as calcium carbonate and magnesium stearate; cellulose derivatives such as lactose, crystalline cellulose, ethyl cellulose, and low-substituted hydroxypropylcellulose; and crosslinked polyvinylpyrrolidone. The filler may be used singly or in combination of two or more. The content of the filler in the adhesive layer is preferably 5 parts by mass or less, and more preferably 0.1 to 2 parts by mass, relative to 100 parts by mass of the acrylic adhesive.

The thickness of the adhesive layer is preferably to 200 μm, more preferably 30 to 150 μm, and particularly preferably 50 to 120 μm. The thickness of the adhesive layer of 20 μm or more can allow the adhesive layer to contain the drug in an amount necessary to obtain a desired medicinal effect. The thickness of the adhesive layer of 200 μm or less can eliminate a strong drying condition at the time of manufacturing in order to reduce the residual solvent in the adhesive layer, and thus can suppress volatilization or decomposition of the drug in the adhesive layer.

[Backing]

In the adhesive patch of the present invention, the adhesive layer is integrally laminated on one side of the backing. The backing is required to prevent loss of the drug in the adhesive layer and have strength to impart self-retentivity to the adhesive patch. Examples of such a backing include resin film, non-woven fabric, woven fabric, knitted fabric, and aluminum sheet.

Examples of the resin constituting the resin film include cellulose acetate, rayon, polyethylene terephthalate, a plasticized vinyl acetate-vinyl chloride copolymer, nylon, an ethylene-vinyl acetate copolymer, a plasticized polyvinyl chloride, polyurethane, polyethylene, polypropylene, and polyvinylidene chloride. Among these, polyethylene terephthalate is preferred because it prevents the loss of a volatile drug from the adhesive layer.

Examples of the material constituting the nonwoven fabric include polyethylene, polypropylene, an ethylene-vinyl acetate copolymer, an ethylene-methyl (meth)acrylate copolymer, nylon, a polyester, vinylon, an SIS copolymer, an SEBS copolymer, rayon, and cotton, with a polyester being preferred. These materials may be used singly or in combination of two or more.

The backing may be a single layer or a laminated sheet in which a plurality of layers are integrally laminated. Examples of the laminated sheet include a laminated sheet in which a polyethylene terephthalate sheet and a nonwoven fabric or a flexible resin film are integrally laminated.

The thickness of the backing is not particularly limited, and is preferably 2 to 200 μm, more preferably 2 to 100 μm.

[Release Liner]

In the adhesive patch of the present invention, a release liner may be integrally laminated on one side of the adhesive layer so as to be freely releasable. The release liner is used to prevent loss of the drug in the adhesive layer and to protect the adhesive layer.

Examples of the release liner include paper and a resin film. Examples of the resin constituting the resin film include a polyethylene terephthalate, polyethylene, polypropylene, polyvinyl chloride, and polyvinylidene chloride. The side of the release liner facing the adhesive layer is preferably subjected to a release treatment.

[Production Method of Adhesive Patch]

Examples of a method for producing the adhesive patch of the present invention include: (1) a method in which an adhesive layer-forming solution, which includes the drug, levulinic acid, the acrylic adhesive, a solvent, and, if necessary, other additives, is applied onto one side of the backing and then dried to remove the solvent, which may lead to integrally laminate an adhesive layer on the one side of the backing, and, if necessary, a release liner is laminated on the adhesive layer such that a release-treated side of the release liner faces the adhesive layer; and (2) a method in which the above-mentioned adhesive layer-forming solution is applied onto the release-treated side of the release liner and then dried to form an adhesive layer on the release liner, and the backing is integrally laminated on the adhesive layer.

The adhesive layer-forming solution can be obtained by uniformly stirring the drug, levulinic acid, the acrylic adhesive and the solvent, and, if necessary, other additives. Examples of the solvent include toluene, normal hexane, cyclohexane, normal heptane, and ethyl acetate. The solvents may be used singly or in combination of two or more.

As described above, the adhesive patch of the present invention includes the adhesive layer that is excellent in adhesiveness, and thus the adhesive patch is preferably used by directly sticking this adhesive layer to the skin.

### Advantageous Effects of Invention

The adhesive patch of the present invention having the configuration described above is excellent in both the percutaneous absorbability of the drug and the adhesiveness to the skin.

### BRIEF DESCRIPTION OF DRAWINGS

FIG. 1 is a graph schematically showing a relationship between a moving distance of a cylindrical probe and a load in probe tack testing.

### DESCRIPTION OF EMBODIMENTS

The present invention will be described in more detail below using examples. However, the present invention is not limited to these examples.

### EXAMPLES

(Preparation of Acrylic Polymer (A1))

A reaction liquid containing monomers including 75 parts by mass of 2-ethylhexyl acrylate and 25 parts by mass of N-vinyl-2-pyrrolidone, and 50 parts by mass of ethyl acetate was supplied to a polymerizer, and the inside of the polymerizer was brought to a nitrogen atmosphere at 80° C. Then, a polymerization initiator solution obtained by dissolving 1.2 parts by mass of lauroyl peroxide in 30 parts by mass of ethyl acetate and 20 parts by mass of cyclohexane was added to the above-mentioned reaction liquid over 24 hours to copolymerize the above-mentioned monomers. After the polymerization was completed, ethyl acetate was further added to the above-mentioned reaction liquid to obtain an acrylic polymer (A1) solution containing 30% by mass of the acrylic polymer (A1).

(Preparation of Acrylic Polymer (A2))

A reaction liquid containing monomers including 40 parts by mass of n-octyl acrylate, 50 parts by mass of ethyl acrylate, and 10 parts by mass of N-vinyl-2-pyrrolidone, and parts by mass of ethyl acetate was supplied to a polymerizer, and the inside of the polymerizer was brought to a nitrogen atmosphere at 80° C. Then, a polymerization initiator solution obtained by dissolving 1 part by mass of lauroyl peroxide in 30 parts by mass of ethyl acetate and 20 parts by mass of cyclohexane was added to the above-mentioned reaction liquid over 24 hours to copolymerize the above-mentioned monomers. After the polymerization was completed, ethyl acetate was further added to the above-mentioned reaction liquid to obtain an acrylic polymer (A2) solution containing 30% by mass of the acrylic polymer (A2).

(Preparation of Acrylic Polymer (A3))

A reaction liquid containing monomers including 13 parts by mass of dodecyl methacrylate, 78 parts by mass of 2-ethylhexyl methacrylate, and 9 parts by mass of 2-ethylhexyl acrylate, and 50 parts by mass of ethyl acetate was supplied to a polymerizer, and the inside of the polymerizer was brought to a nitrogen atmosphere at 80° C. Then, a polymerization initiator solution obtained by dissolving 0.5 parts by mass of benzoyl peroxide in 10 parts by mass of ethyl acetate and 10 parts by mass of cyclohexane was added to the above-mentioned reaction liquid over 24 hours to copolymerize the above-mentioned monomers. After the polymerization was completed, ethyl acetate was further added to the above-mentioned reaction liquid to obtain an acrylic polymer (A3) solution containing 30% by mass of the acrylic polymer (A3).

(Preparation of Acrylic Polymer (A4))

A reaction liquid containing monomers including 71 parts by mass of 2-ethylhexyl acrylate, 24 parts by mass of methyl acrylate, and 5 parts by mass of 2-hydroxyethyl acrylate, and parts by mass of ethyl acetate was supplied to a polymerizer, and the inside of the polymerizer was brought to a nitrogen atmosphere at 80° C. Then, a polymerization initiator solution obtained by dissolving 0.5 parts by mass of benzoyl peroxide in 10 parts by mass of n-hexane was added to the above-mentioned reaction liquid over 24 hours to copolymerize the above-mentioned monomers. After the polymerization was completed, ethyl acetate was further added to the above-mentioned reaction liquid to obtain an acrylic polymer (A4) solution containing 40% by mass of the acrylic polymer (A4).

(Preparation of Acrylic Polymer (A5))

A reaction liquid containing monomers including 50 parts by mass of n-octyl acrylate, 45 parts by mass of ethyl acrylate, and 5 parts by mass of N-vinyl-2-pyrrolidone, and 140 parts by mass of ethyl acetate was supplied to a polymerizer, and the inside of the polymerizer was brought to a nitrogen atmosphere at 80° C. Then, a polymerization initiator solution obtained by dissolving 0.4 parts by mass of lauroyl peroxide in 20 parts by mass of ethyl acetate was added to the above-mentioned reaction liquid over 14 hours to copolymerize the above-mentioned monomers. After the polymerization was completed, ethyl acetate was further added to the above-mentioned reaction liquid to obtain an acrylic polymer (A5) solution containing 30% by mass of the acrylic polymer (A5).

(Preparation of Acrylic Polymer (A6))

A reaction liquid containing monomers including 65 parts by mass of 2-ethylhexyl acrylate and 35 parts by mass of N-vinyl-2-pyrrolidone, and 185 parts by mass of ethyl acetate was supplied to a polymerizer, and the inside of the polymerizer was brought to a nitrogen atmosphere at 80° C. Then, a polymerization initiator solution obtained by dissolving 0.6 parts by mass of lauroyl peroxide in 17 parts by mass of ethyl acetate was added to the above-mentioned reaction liquid over 14 hours to copolymerize the above-mentioned monomers. After the polymerization was completed, ethyl acetate was further added to the above-mentioned reaction liquid to obtain an acrylic polymer (A6) solution containing 30% by mass of the acrylic polymer (A6).

(Preparation of Acrylic Polymer (A7))

A reaction liquid containing monomers including 75 parts by mass of 2-ethylhexyl acrylate, 3 parts by mass of acrylic acid, and 22 parts by mass of N-vinyl-2-pyrrolidone, and 150 parts by mass of ethyl acetate was supplied to a polymerizer, and the inside of the polymerizer was brought to a nitrogen atmosphere at 80° C. Then, a polymerization initiator solution obtained by dissolving 0.6 parts by mass of lauroyl peroxide in 17 parts by mass of ethyl acetate was added to the above-mentioned reaction liquid over 14 hours to copolymerize the above-mentioned monomers. After the polymerization was completed, ethyl acetate was further added to the above-mentioned reaction liquid to obtain an acrylic polymer (A7) solution containing 30% by mass of the acrylic polymer (A7).

Solubility parameters of the monomers used for preparing the acrylic polymers (A1) to (A7) are shown in Table 1.

TABLE 1

| | Solubility parameter |
|---|---|
| | (cal/cm³)^{1/2} |
| Ethyl acrylate | 8.8 |
| 2-Ethylhexyl acrylate | 7.8 |
| 2-Ethylhexyl methacryate | 8.3 |
| n-Octyl acrylate | 8.2 |
| Dodecyl methacrylate | 8.2 |
| Methyl acrylate | 9.4 |
| 2-Hydroxyethyl acrylate | 12.1 |
| N-Vinyl-2-pyrrolidone | 9.7 |
| Acrylic acid | 11.7 |

### Example 1

A free base form blonanserin, levulinic acid, and the acrylic polymer (A1) solution were mixed such that the free base form blonanserin, levulinic acid, and the acrylic polymer (A1) were each comprised in an adhesive layer in a blending amount shown in Table 2, thereby producing an adhesive layer-forming solution. Next, a polyethylene terephthalate film having a thickness of 38 μm and subjected to a silicone release treatment was prepared as a release liner. The adhesive layer-forming solution was applied onto the silicone release-treated side of this polyethylene terephthalate film and dried at 60° C. for 30 minutes, thereby producing a laminate with an 80 μm-thick adhesive layer formed on the silicone release-treated side of the polyethylene terephthalate film. Then, a polyethylene terephthalate film with a thickness of 25 μm was prepared as a backing, and one side of the backing and the adhesive layer of the above-mentioned laminate were laminated so as to face each other, so that the adhesive layer of the laminate was transferred to and integrally laminated on the backing, thereby producing an adhesive patch. The gel fraction of the adhesive layer is shown in Table 2.

Note that 0.1 g or more of the free base form blonanserin could be dissolved in 1 mL of levulinic acid at a liquid temperature of 35° C.

### Example 2

An adhesive patch was produced in the same manner as in Example 1 except that the free base form blonanserin, levulinic acid, and the acrylic polymer (A2) solution were mixed such that the free base form blonanserin, levulinic acid, and the acrylic polymer (A2) were each comprised in the adhesive layer in a blending amount shown in Table 2, thereby producing an adhesive layer-forming solution. The gel fraction of the adhesive layer is shown in Table 2.

### Example 3

An adhesive patch was produced in the same manner as in Example 1 except that the free base form blonanserin, levulinic acid, and the acrylic polymer (A4) solution were mixed such that the free base form blonanserin, levulinic acid, and the acrylic polymer (A4) were each comprised in the adhesive layer in a blending amount shown in Table 2, thereby producing an adhesive layer-forming solution. The gel fraction of the adhesive layer is shown in Table 2.

### Comparative Example 1

An adhesive patch was produced in the same manner as in Example 1 except that the free base form blonanserin, isopropyl myristate, and the acrylic polymer (A2) solution were mixed such that the free base form blonanserin, isopropyl myristate, and the acrylic polymer (A2) were each comprised in the adhesive layer in a blending amount shown in Table 2, thereby producing an adhesive layer-forming solution. The gel fraction of the adhesive layer is shown in Table 2.

### Comparative Example 2

An adhesive patch was produced in the same manner as in Example 1 except that the free base form blonanserin, levulinic acid, and the acrylic polymer (A3) solution were mixed such that the free base form blonanserin, levulinic acid, and the acrylic polymer (A3) were each comprised in the adhesive layer in a blending amount shown in Table 2, thereby producing an adhesive layer-forming solution. The gel fraction of the adhesive layer is shown in Table 2. However, in the adhesive patch of Comparative example 2, a liquid component, which was considered to be levulinic acid, was leaked excessively to the surface of the adhesive layer immediately after production, resulting in a decrease in the adhesiveness of the adhesive layer. Thus, the adhesive patch of Comparative example 2 was not evaluated for the percutaneous absorbability described below.

### Example 4

A free base form guanfacine, levulinic acid, isopropyl myristate, and the acrylic polymer (A2) solution were mixed such that the free base form guanfacine, levulinic acid, isopropyl myristate, and the acrylic polymer (A2) were each comprised in the adhesive layer in a blending amount shown in Table 3, thereby producing an adhesive layer-forming solution. Next, a polyethylene terephthalate film having a thickness of μm and subjected to a silicone release treatment was prepared as a release liner. The adhesive layer-forming solution was applied onto the silicone release-treated side of this polyethylene terephthalate film and dried at 60° C. for 30 minutes, thereby producing a laminate with a 70 μm-thick adhesive layer formed on the silicone release-treated side of the polyethylene terephthalate film. Then, a polyethylene terephthalate film with a thickness of 38 μm was prepared as a backing, and one side of the backing and the adhesive layer of the above-mentioned laminate were laminated so as to face each other, so that the adhesive layer of the laminate was transferred to and integrally laminated on the backing, thereby producing an adhesive patch. The gel fraction of the adhesive layer is shown in Table 3.

Note that 0.1 g or more of the free base form guanfacine could be dissolved in 1 mL of levulinic acid at a liquid temperature of 35° C.

### Comparative Example 3

An adhesive patch was produced in the same manner as in Example 4 except that the free base form guanfacine, isopropyl myristate, and the acrylic polymer (A2) solution were mixed such that the free base form guanfacine, isopropyl myristate, and the acrylic polymer (A2) were each comprised in the adhesive layer in a blending amount shown in Table 3, thereby producing an adhesive layer-forming solution. The gel fraction of the adhesive layer is shown in Table 3.

Note that in Table 3, the blending amount of each of the free base form guanfacine, levulinic acid, isopropyl myristate, and the acrylic polymer (A2) in the adhesive layer is indicated by a numerical value not enclosed in parentheses. Furthermore, the content ratio of each of the free base form guanfacine, levulinic acid, and the acrylic polymer (A2) in the adhesive layer in 100 parts by mass of the total amount of the free base form guanfacine, levulinic acid, and the acrylic polymer (A2) is indicated by a numerical value enclosed in parentheses in the column of each component in Table 3. Furthermore, the content of isopropyl myristate in the adhesive layer relative to 100 parts by mass of the acrylic polymer (A2) is indicated by a numerical value enclosed in parentheses in the column of “Isopropyl myristate” in Table 3.

### Example 5

A free base form rotigotine, levulinic acid, isopropyl myristate, and the acrylic polymer (A2) solution were mixed such that the free base form rotigotine, levulinic acid, isopropyl myristate, and the acrylic polymer (A2) were each comprised in the adhesive layer in a blending amount shown in Table 4, thereby producing an adhesive layer-forming solution. Next, a polyethylene terephthalate film having a thickness of 38 μm and subjected to a silicone release treatment was prepared as a release liner. The adhesive layer-forming solution was applied onto the silicone release-treated side of this polyethylene terephthalate film and dried at 60° C. for 30 minutes, thereby producing a laminate with a 45 μm-thick adhesive layer formed on the silicone release-treated side of the polyethylene terephthalate film. Then, a polyethylene terephthalate film with a thickness of 25 μm was prepared as a backing, and one side of the backing and the adhesive layer of the above-mentioned laminate were laminated so as to face each other, so that the adhesive layer of the laminate was transferred to and integrally laminated on the backing, thereby producing an adhesive patch. The gel fraction of the adhesive layer is shown in Table 4.

Note that 0.1 g or more of the free base form rotigotine could be dissolved in 1 mL of levulinic acid at a liquid temperature of 35° C.

### Comparative Example 4

An adhesive patch was produced in the same manner as in Example 5 except that the free base form rotigotine, isopropyl myristate, and the acrylic polymer (A2) solution were mixed such that the free base form rotigotine, isopropyl myristate, and the acrylic polymer (A2) were each comprised in the adhesive layer in a blending amount shown in Table 4, thereby producing an adhesive layer-forming solution. The gel fraction of the adhesive layer is shown in Table 4.

### Comparative Example 5

An adhesive patch was produced in the same manner as in Example 5 except that the free base form rotigotine, levulinic acid, isopropyl myristate, and the acrylic polymer (A3) solution were mixed such that the free base form rotigotine, levulinic acid, isopropyl myristate, and the acrylic polymer (A3) were each comprised in the adhesive layer in a blending amount shown in Table 4, thereby producing an adhesive layer-forming solution. However, in the adhesive patch of Comparative example 5, a liquid component, which was considered to be levulinic acid, was leaked excessively to the surface of the adhesive layer immediately after production, resulting in a decrease in the adhesiveness of the adhesive layer. Thus, the adhesive patch of Comparative example 5 was not evaluated for the percutaneous absorbability described below. The gel fraction of the adhesive layer is shown in Table 4.

### Examples 6 to 16 and Comparative Examples 6 to 8

Adhesive patches were produced in the same manner as in Example 5 except that the free base form rotigotine, levulinic acid, isopropyl myristate, the acrylic polymer (A1) solution to the acrylic polymer (A3) solution, and the acrylic polymer (A5) solution to the acrylic polymer (A7) solution were mixed such that the free base form rotigotine, levulinic acid, isopropyl myristate, the acrylic polymer (A1) to the acrylic polymer (A3), and the acrylic polymer (A5) to the acrylic polymer (A7) were each comprised in the adhesive layers in a blending amount shown in Table 4, thereby producing adhesive layer-forming solutions. The gel fraction of the adhesive layers is shown in Table 4.

Note that in Table 4, the blending amount of each of the free base form rotigotine, levulinic acid, isopropyl myristate, and the acrylic polymer (A) in the adhesive layer is indicated by a numerical value not enclosed in parentheses. Furthermore, the content ratio of each of the free base form rotigotine, levulinic acid, and the acrylic polymer (A) in the adhesive layer in 100 parts by mass of the total amount of the free base form rotigotine, levulinic acid, and the acrylic polymer (A) is indicated by a numerical value enclosed in parentheses in the column of each component in Table 4. Furthermore, the content of isopropyl myristate in the adhesive layer relative to 100 parts by mass of the acrylic polymer (A) is indicated by a numerical value enclosed in parentheses in the column of “isopropyl myristate” in Table 4.

(Performance Evaluation of Adhesive Patch)

The adhesive patches of Examples and Comparative examples were evaluated for the percutaneous absorbability according to the following procedure. Furthermore, the adhesive patches of Examples and Comparative examples were evaluated for the adhesiveness according to the following procedure.

(Percutaneous Absorbability)

Five flat square test pieces, each with an area of 3 cm², were punched out from the adhesive patch. The adhesive layer of each of the three test pieces was dissolved in a solvent, and the amount of the drug was measured using HPLC (high-performance liquid chromatography). The arithmetic mean value of these measurements was calculated as a “pre-test drug amount (μg/cm²)”. The release liner was peeled off from each of the remaining two test pieces to expose the entire adhesive layer. Then, each test piece was stuck to the back of a Wistar rat (7 weeks old), whose back was shaved, for 24 hours. After removing the test piece from the back of the rat, each adhesive layer was dissolved in a solvent, and the amount of the drug was measured using HPLC. The arithmetic mean value of these measurements was calculated as a “post-test drug amount (μg/cm²)”. A value obtained by subtracting the “post-test drug amount” from the “pre-test drug amount” was defined as a “percutaneous absorption amount (μg/cm²)” and shown in Tables 2 to 4.

As the solvent for dissolving the adhesive layer in the measurement of the drug amount using HPLC, tetrahydrofuran was used in a case where the drug included in the adhesive layer was the free base form blonanserin or the free base form rotigotine, while dimethylformamide was used in a case where the drug included in the adhesive layer was the free base form guanfacine.

(Adhesiveness Test)

A test piece (1.7 mm height×1.7 mm width) was cut out from the adhesive patch. The release liner was peeled off and removed from the test piece to expose the adhesive layer, and the test piece was placed on a horizontal surface with the adhesive layer facing up. A cylindrical probe was brought into contact with the surface of the adhesive layer according to “3.4. Probe tack testing” of General Tests “6.13 Methods of Adhesion Testing” of the Japanese Pharmacopoeia, 17th Edition. Then, the cylindrical probe was peeled off while being moved vertically from the surface of the adhesive layer, and the resistance force received by the cylindrical probe due to the adhesive force of the surface of the adhesive layer when peeling off was measured as a load (N/cm²). Note that the load measurement was performed multiple times while changing the measurement interval for each moving distance of the cylindrical probe in contact with the adhesive layer surface.

Specifically, the load was measured each time the cylindrical probe moved by 0.01 mm until the moving distance of the cylindrical probe in contact with the adhesive layer surface reached 0.12 mm (number of measurements n: 1st to 12th). The load was measured each time the cylindrical probe moved by 0.02 mm until the moving distance of the cylindrical probe exceeded 0.12 mm and reached 0.48 mm (number of measurements n: 13th to 30th). The load was measured each time the cylindrical probe moved by 0.03 mm until the moving distance of the cylindrical probe exceeded 0.48 mm and reached 1.02 mm (number of measurements n: 31st to 48th). The load was measured each time the cylindrical probe moved by 0.04 mm until the moving distance of the cylindrical probe exceeded 1.02 mm and reached 1.98 mm (number of measurements n: 49th to 72nd). After that, the load was measured each time the cylindrical probe moved by 0.05 mm (number of measurements n: 73rd and thereafter). Then, the maximum load (N/cm²) and the load area [(N/cm²)×mm] required to peel off the cylindrical probe are shown in Tables 2 to 4.

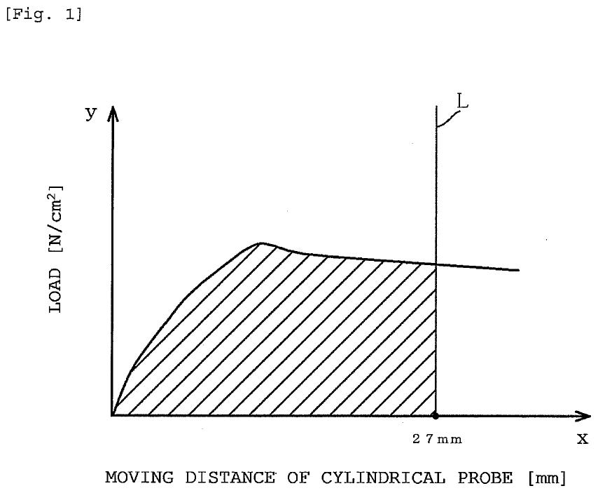

Note that the load area was calculated on the basis of the following formula (1) where Ln (N/cm²) represented the load measured at the n-th time in the load measured until the moving distance of the cylindrical probe in contact with the adhesive layer surface reached 27 mm. Note that the load area is described below. First, the measured values of the load measured as described above were plotted on a graph with the X-axis representing the moving distance (mm) of the cylindrical probe and the Y-axis representing the load (N/cm²), thereby drawing a curve. A schematic diagram of the above-mentioned graph is shown in FIG. 1. In the above-mentioned graph, the load area is represented by a value approximated to the area of a part (shaded part in FIG. 1) surrounded by the above-mentioned curve, the X axis, and a straight line L that passes through the point on the X axis at X=27 mm and is parallel to the Y axis. That is, the load area corresponds to an integrated value of the load until the moving distance of the cylindrical probe in contact with the adhesive layer surface reaches 27 mm.

$$[ Mathematical formula 1 ]$$

$$Load Area [ ( N / {cm}^{2} ) \times mm ] = {\sum }_{n = 1}^{12} [ \frac{( {L}_{n} + {L}_{n - 1} )}{2} ] \times 0.01 + {\sum }_{n = 13}^{30} [ \frac{( {L}_{n} + {L}_{n - 1}}{2} ] \times 0.02 + {\sum }_{n = 31}^{48} [ \frac{( {L}_{n} + {L}_{n - 1} )}{2} ] \times 0.03 + {\sum }_{n = 49}^{72} [ \frac{( {L}_{n} + {L}_{n - 1} )}{2} ] \times 0.04 + {\sum }_{n = 73}^{574} [ \frac{( {L}_{n} + {L}_{n - 1} )}{2} ] \times 0.05 \qquad ( 1 )$$

The maximum load in the probe tack testing is preferably 23 N/cm² or more. The maximum load of 23 N/cm² or more means that the adhesive layer exhibits excellent adhesiveness, allowing for reduction of peeling of the adhesive patch from the skin during application and stably stick the adhesive patch to the skin. Note that if the maximum load is too high, the skin may be damaged when the adhesive patch is removed from the skin. Thus, the maximum load in the probe tack testing is preferably 100 N/cm² or less.

Furthermore, the load area in the probe tack testing is preferably 20 (N/cm²)×mm or more. The load area of 20 (N/cm²)×mm or more means that the adhesive layer exhibits further excellent adhesiveness, allowing for further reduction of the adhesive patch peeling off of the skin during application and further stability in the adhesive patch sticking to the skin. Note that if the load area is too high, the skin may be damaged when the adhesive patch is removed from the skin. Thus, the load area in the probe tack testing is preferably 120 (N/cm²)×mm or less.

TABLE 2

| | | | | | Comparative | Comparative |
|---|---|---|---|---|---|---|
| | | Example 1 | Example 2 | Example 3 | Example 1 | Example 2 |
| Adhesive layer | Free base form blonanserin | 6.5 | 6.5 | 6.5 | 6.5 | 6.5 |
| composition | Levulinic acid | 10 | 10 | 10 | 0 | 10 |
| [Parts by mass] | Isopropyl myristate | 0 | 0 | 0 | 10 | 0 |
| | Acrylic polymer (A1) | 83.5 | 0 | 0 | 0 | 0 |
| | Acrylic polymer (A2) | 0 | 83.5 | 0 | 83.5 | 0 |
| | Acrylic polymer (A3) | 0 | 0 | 0 | 0 | 83.5 |
| | Acrylic polymer (A4) | 0 | 0 | 83.5 | 0 | 0 |

| | | | | | |
|---|---|---|---|---|---|
| Mass ratio [(mass of levulinic acid)/(mass of drug)] | 1.5 | 1.5 | 1.5 | — | 1.5 |
| Gel fraction of adhesive layer [% by mass] | 1 or less | 1 or less | 1 or less | 1 or less | 1 or less |
| Percutaneous absorption amount [μg/cm²] | 102 | 98 | 102 | 46 | — |
| Probe tack testing maximum load [N/cm²] | 74 | 57 | 40 | 50 | 19 |
| Probe tack testing load area [(N/cm²) × mm] | 94 | 80 | 48 | 57 | 8 |

TABLE 3

| | | | | Comparative | |
|---|---|---|---|---|---|
| | | Example 4 | | example 3 | |
| Adhesive | Free base form | 5 | (5.6) | 5 | (5.6) |
| layer | guanfacine | | | | |
| composition | Levulinic | 2.6 | (2.9) | 0 | (0) |
| [Parts by | acid | | | | |
| mass] | Isopropyl myristate | 10 | (12.1) | 10 | (11.8) |
| | Acrylic polymer (A2) | 82.4 | (91.5) | 85 | (94.4) |

| | | |
|---|---|---|
| Mass ratio [(mass of | 0.5 | — |
| levulinic acid)/(mass of drug)] | | |
| Gel fraction of adhesive layer | 1 or less | 1 or less |
| [% by mass] | | |
| Percutaneous absorption amount | 38 | 0 |
| [(μg/cm²] | | |
| Probe tack testing maximum load | 39 | 36 |
| [N/cm²] | | |
| Probe tack testing load area | 37 | 28 |
| [(N/cm²)xmm] | | |

TABLE 4

| | | Exam- | Exam- | Exam- | Exam- | Exam- | Exam- | Exam- | Exam- | Exam- | Exam- |
|---|---|---|---|---|---|---|---|---|---|---|---|
| | | ple 5 | ple 6 | ple 7 | ple 8 | ple 9 | ple 10 | ple 11 | ple 12 | ple 13 | ple 14 |
| Adhesive layer | Free base form | 10 | 25 | 25 | 25 | 25 | 25 | 20 | 25 | 25 | 25 |
| composition | rotigotine | (13.2) | (28.4) | (28.4) | (28.4) | (28.4) | (28.4) | (22.7) | (28.4) | (28.4) | (25.0) |
| [Parts by | Levulinic acid | 10 | 10 | 10 | 10 | 10 | 10 | 8 | 5 | 15 | 10 |
| mass] | | (13.2) | (11.4) | (11.4) | (11.4) | (11.4) | (11.4) | (9.1) | (5.7) | (17.1) | (10.0) |
| | Isopropyl myristate | 24 | 12 | 12 | 12 | 12 | 12 | 12 | 12 | 12 | 0 |
| | | (42.9) | (22.6) | (22.6) | (22.6) | (22.6) | (22.6) | (20.0) | (20.7) | (25.0) | (0) |
| | Acrylic polymer (A2) | 56 | 53 | 0 | 0 | 0 | 0 | 60 | 58 | 48 | 65 |
| | | (73.6) | (60.2) | (0) | (0) | (0) | (0) | (68.2) | (65.9) | (54.5) | (65.0) |
| | Acrylic polymer (A5) | 0 | 0 | 53 | 0 | 0 | 0 | 0 | 0 | 0 | 0 |
| | | (0) | (0) | (60.2) | (0) | (0) | (0) | (0) | (0) | (0) | (0) |
| | Acrylic polymer (A1) | 0 | 0 | 0 | 53 | 0 | 0 | 0 | 0 | 0 | 0 |
| | | (0) | (0) | (0) | (60.2) | (0) | (0) | (0) | (0) | (0) | (0) |
| | Acrylic polymer (A6) | 0 | 0 | 0 | 0 | 53 | 0 | 0 | 0 | 0 | 0 |
| | | (0) | (0) | (0) | (0) | (60.2) | (0) | (0) | (0) | (0) | (0) |
| | Acrylic polymer (A7) | 0 | 0 | 0 | 0 | 0 | 53 | 0 | 0 | 0 | 0 |
| | | (0) | (0) | (0) | (0) | (0) | (60.2) | (0) | (0) | (0) | (0) |
| | Acrylic polymer (A3) | 0 | 0 | 0 | 0 | 0 | 0 | 0 | 0 | 0 | 0 |
| | | (0) | (0) | (0) | (0) | (0) | (0) | (0) | (0) | (0) | (0) |

| | | | | | | | | | | |
|---|---|---|---|---|---|---|---|---|---|---|
| Mass ratio [(mass of levulinic acid)/ | 1.0 | 0.4 | 0.4 | 0.4 | 0.4 | 0.4 | 0.4 | 0.2 | 0.6 | 0.4 |
| (mass of drug)] | | | | | | | | | | |
| Gel fraction of adhesive layer | 1 or | 1 or | 1 or | 1 or | 1 or | 1 or | 1 or | 1 or | 1 or | 1 or |
| [% by mass] | less | less | less | less | less | less | less | less | less | less |
| Percutaneous absorption amout | 152 | 250 | 203 | 163 | 160 | 149 | 182 | 191 | 219 | 155 |
| [μg/cm²] | | | | | | | | | | |
| Probe tack testing maximum load | 25 | 33 | 36 | 38 | 34 | 39 | 37 | 36 | 33 | 40 |
| [N/cm²] | | | | | | | | | | |
| Probe tack testing load area | 32 | 25 | 24 | 28 | 22 | 26 | 27 | 27 | 23 | 27 |
| [N/cm²) × mm] | | | | | | | | | | |

| | | | | Compar- | Compar- | Compar- | Compar- | Compar- |
|---|---|---|---|---|---|---|---|---|
| | | Exam- | Exam- | ative | ative | ative | ative | ative |
| | | ple 15 | ple 16 | example 4 | example 5 | example 6 | example 7 | example 8 |
| Adhesive layer | Free base form | 25 | 30 | 10 | 10 | 25 | 25 | 25 |
| composition | rotigotine | (26.0) | (34.1) | (13.2) | (13.2) | (28.4) | (28.4) | (28.4) |
| [Parts by | Levulinic acid | 10 | 12 | 0 | 10 | 0 | 0 | 0 |
| mass] | | (10.5) | (13.6) | (0) | (13.2) | (0) | (0) | (0) |
| | Isopropyl myristate | 4 | 12 | 24 | 24 | 12 | 12 | 12 |
| | | (6.6) | (26.1) | (36.4) | (42.9) | (19.0) | (19.0) | (19.0) |
| | Acrylic polymer (A2) | 61 | 46 | 66 | 0 | 63 | 0 | 0 |
| | | (63.5) | (52.3) | (86.8) | (0) | (71.6) | (0) | (0) |
| | Acrylic polymer (A5) | 0 | 0 | 0 | 0 | 0 | 0 | 0 |
| | | (0) | (0) | (0) | (0) | (0) | (0) | (0) |
| | Acrylic polymer (A1) | 0 | 0 | 0 | 0 | 0 | 63 | 0 |
| | | (0) | (0) | (0) | (0) | (0) | (71.6) | (0) |
| | Acrylic polymer (A6) | 0 | 0 | 0 | 0 | 0 | 0 | 0 |
| | | (0) | (0) | (0) | (0) | (0) | (0) | (0) |
| | Acrylic polymer (A7) | 0 | 0 | 0 | 0 | 0 | 0 | 63 |
| | | (0) | (0) | (0) | (0) | (0) | (0) | (71.6) |
| | Acrylic polymer (A3) | 0 | 0 | 0 | 56 | 0 | 0 | 0 |
| | | (0) | (0) | (0) | (73.6) | (0) | (0) | (0) |

| | | | | | | | |
|---|---|---|---|---|---|---|---|
| Mass ratio [(mass of levulinic acid)/ | 0.4 | 0.4 | — | 1.0 | — | — | — |
| (mass of drug)] | | | | | | | |
| Gel fraction of adhesive layer | 1 or | 1 or | 1 or | 1 or | 1 or | 1 or | 1 or |
| [% by mass] | less | less | less | less | less | less | less |
| Percutaneous absorption amout | 238 | 253 | 114 | — | 112 | 59 | 53 |
| [μg/cm²] | | | | | | | |
| Probe tack testing maximum load | 42 | 37 | 34 | 20 | 23 | 32 | 22 |
| [N/cm²] | | | | | | | |
| Probe tack testing load area | 31 | 28 | 36 | 17 | 14 | 20 | 12 |
| [N/cm²) × mm] | | | | | | | |

### INDUSTRIAL APPLICABILITY

According to the present invention, it is possible to provide an adhesive patch that is excellent in both percutaneous absorbability of a drug and adhesiveness to the skin.

### CROSS-REFERENCE TO RELATED APPLICATION

The present application claims the priority under Japanese Patent Application No. 2020-138958, filed on Aug. 19, 2020, the disclosure of which is hereby incorporated in its entirety by reference.

1. An adhesive patch comprising: a backing; and an adhesive layer integrally laminated on one side of the backing,
the adhesive layer including a drug, levulinic acid, and an acrylic adhesive including an acrylic polymer (A) containing a vinyl-based monomer (I) moiety having a solubility parameter of 9 (cal/cm³)² or more.

2. The adhesive patch according to claim 1, wherein the drug includes an amine structure in a molecular structure.

3. The adhesive patch according to claim 1, wherein the drug includes at least one selected from the group consisting of blonanserin, buprenorphine, rotigotine, and guanfacine.

4. The adhesive patch according to claim 1, wherein a mass ratio of levulinic acid to the drug [(mass of levulinic acid)/(mass of drug)] is 0.2 or more and 4 or less.

5. The adhesive patch according to claim 1, wherein the vinyl-based monomer (I) moiety contains at least one selected from the group consisting of an N-vinyl-2-pyrrolidone moiety, a diacetone acrylamide moiety, a methyl acrylate moiety, and a 2-hydroxyethyl acrylate moiety.

6. The adhesive patch according to claim 1, wherein the acrylic polymer (A) contains a vinyl-based monomer (II) moiety having a solubility parameter of less than 9 (cal/cm³)^{1/2}.

7. The adhesive patch according to claim 6, wherein the vinyl-based monomer (II) moiety contains an alkyl (meth)acrylate moiety having an alkyl group having 1 to 16 carbon atoms.

8. The adhesive patch according to claim 6, wherein the vinyl-based monomer (II) moiety contains at least one selected from the group consisting of an ethyl acrylate moiety, a 2-ethylhexyl acrylate moiety, a 2-ethylhexyl methacrylate moiety, an n-octyl acrylate moiety, and a dodecyl methacrylate moiety.

9. The adhesive patch according to claim 6, wherein:
the drug includes at least one selected from the group consisting of blonanserin, buprenorphine, rotigotine, and guanfacine;
the vinyl-based monomer (I) moiety contains at least one selected from the group consisting of an N-vinyl-2-pyrrolidone moiety, a diacetone acrylamide moiety, a methyl acrylate moiety, a 2-hydroxyethyl acrylate moiety, and an acrylic acid moiety; and
the vinyl-based monomer (II) moiety contains at least one selected from the group consisting of an ethyl acrylate moiety, a 2-ethylhexyl acrylate moiety, a 2-ethylhexyl methacrylate moiety, an n-octyl acrylate moiety, and a dodecyl methacrylate moiety.
